(19) United States
(12) Patent Application Publication
(10) Pub. No.: US 2025/0319261 A1
(43) Pub. Date: Oct. 16, 2025

(54) DEVICES, SYSTEMS, AND METHODS FOR DRUG AUTHENTICATION AND ADMINISTRATION

(71) Applicant: Adjutant Solutions Group LLC, Indianapolis, IN (US)

(72) Inventors: Mohammad S. Ahmed, Kildeer, IL (US); Benjamin R. Hansen, Greenwood, IN (US); Douglas Koeneman, Carmel, IN (US)

(73) Assignee: Adjutant Solutions Group LLC, Indianapolis, IN (US)

(21) Appl. No.: 19/175,889

(22) Filed: Apr. 10, 2025

Related U.S. Application Data
(60) Provisional application No. 63/632,276, filed on Apr. 10, 2024.

(51) Int. Cl. A61M 5/315; A61M 5/50; G16H 20/13

(52) U.S. Cl. CPC A61M 5/31571; A61M 5/5086; G16H 20/13; A61M 2205/3584; A61M 2205/6009

(57) ABSTRACT

Devices, systems, and methods for authenticating medicines prior to administration within the body. An exemplary medicine administration device includes a receptacle configured to receive a medicine container, a delivery mechanism configured to deliver a medicine from the medicine container to a mammalian recipient, and a processor or control unit in communication with a database, the database configured to store information regarding one or more medicines, wherein the processor or control unit is configured to access said stored information, wherein the device, upon receipt of the medicine container and authentication of the medicine container, is configured to deliver a dose of a medicine from the medicine container via the delivery mechanism.

### PRIORITY

The present application is related to, and claims the priority benefit of, U.S. provisional patent application Ser. No. 63/632,276, filed Apr. 10, 2024, the contents of which are incorporated herein directly and by reference in their entirety.

### BRIEF SUMMARY

The present disclosure includes disclosure of devices, systems, and methods for authenticating medicines prior to administration within the body.

The present disclosure includes disclosure of a medicine administration device, comprising a receptacle configured to receive a medicine container, a delivery mechanism configured to deliver a medicine from the medicine container to a mammalian recipient, and a processor or control unit in communication with a database, the database configured to store information regarding one or more medicines, wherein the processor or control unit is configured to access said stored information, wherein the device, upon receipt of the medicine container and authentication of the medicine container, is configured to deliver a dose of a medicine from the medicine container via the delivery mechanism.

The present disclosure includes disclosure of a medicine administration device, wherein said stored information comprises information selected from the group consisting of a medicine manufacturer, a medicine name, a medicine dosage, a date of medicine manufacture, a medicine lot number, and medicine recall information.

The present disclosure includes disclosure of a medicine administration device, operable to deliver a dose of the medicine from the medicine container when at least one type of information is used to authenticate the medicine container.

The present disclosure includes disclosure of a medicine administration device, operable to deliver a dose of the medicine from the medicine container when two types of information are used to authenticate the medicine container.

The present disclosure includes disclosure of a medicine administration device, operable to deliver a dose of the medicine from the medicine container when three or more types of information are used to authenticate the medicine container.

The present disclosure includes disclosure of a medicine administration device, wherein the database is contained within the device.

The present disclosure includes disclosure of a medicine administration device, wherein the database can be updated via a network in communication with an external database.

The present disclosure includes disclosure of a medicine administration device, wherein the database is contained within an external system, the database accessible by the device via a network.

The present disclosure includes disclosure of a medicine administration device, wherein the medicine administration device is configured to communicate wirelessly with the database contained within the external system.

The present disclosure includes disclosure of a medicine administration device, wherein if the medicine container is not authenticated, the device would not be operable to deliver the dose of the medicine from the medicine container unless a user of the device bypasses authentication.

The present disclosure includes disclosure of a medicine administration device, wherein the processor or control unit is configured to authenticate the medicine container.

The present disclosure includes disclosure of a method of medicine delivery, comprising positioning a medicine container within the receptacle of an exemplary medicine administration device of the present disclosure, and performing authentication of the medicine container.

The present disclosure includes disclosure of a method of medicine delivery, further comprising administering medicine from the medicine container after the medicine container is authenticated.

The present disclosure includes disclosure of a method of medicine delivery, wherein if the medicine container is not authenticated, the device would not be operable to deliver the dose of the medicine from the medicine container unless a user of the device bypasses authentication.

The present disclosure includes disclosure of a method of medicine delivery, wherein if the step of performing authentication fails to authenticate the medicine container, the device is not operable to deliver the medicine from the medicine container.

The present disclosure includes disclosure of a method of medicine delivery, wherein if the step of performing authentication fails to authenticate the medicine container, the device is operable to deliver the medicine from the medicine container if a bypass step is performed.

### BRIEF DESCRIPTION OF THE DRAWINGS

The disclosed embodiments and other features, advantages, and disclosures contained herein, and the matter of attaining them, will become apparent and the present disclosure will be better understood by reference to the following description of various exemplary embodiments of the present disclosure taken in conjunction with the accompanying drawings, wherein:

FIG. 1 shows a block diagram of a system, according to an exemplary embodiment of the present disclosure; and

FIG. 2 shows a block diagram of elements of a medicine administration device, according to an exemplary embodiment of the present disclosure.

As such, an overview of the features, functions and/or configurations of the components depicted in the various figures will now be presented. It should be appreciated that not all of the features of the components of the figures are necessarily described and some of these non-discussed features (as well as discussed features) are inherent from the figures themselves. Other non-discussed features may be inherent in component geometry and/or configuration. Furthermore, wherever feasible and convenient, like reference numerals are used in the figures and the description to refer to the same or like parts or steps. The figures are in a simplified form and not to precise scale.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended.

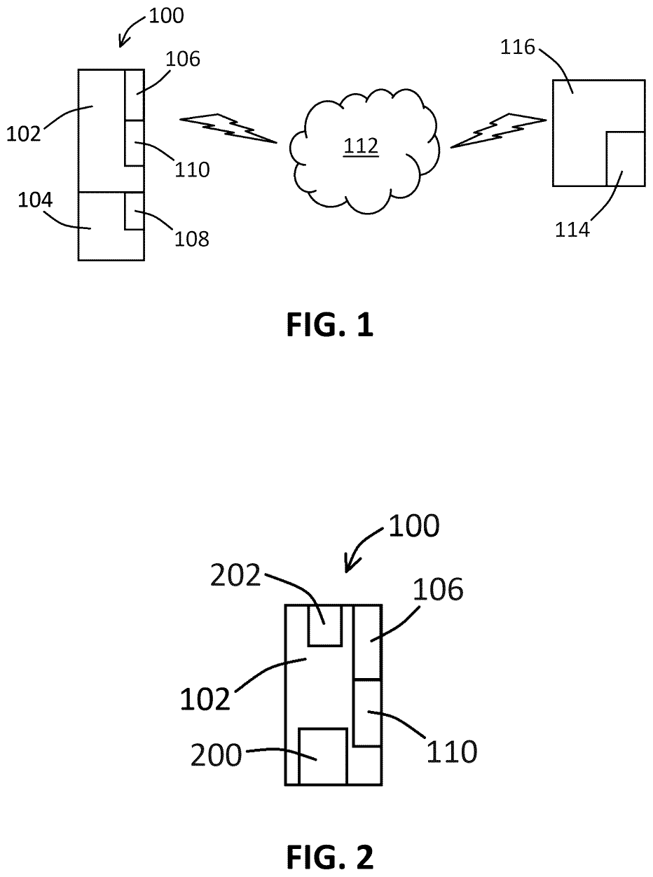

An exemplary system of the present disclosure is shown in block diagram format in FIG. 1. As shown therein, an exemplary system 100 comprises a medicine administration device 102 configured to administer a medicine within the body. Administration devices 102 may be medicine injectors, for example, namely those having one or more needles configured to inject a medicine from within a medicine container 104 therethrough into a mammalian body.

Systems 100 of the present disclosure facilitate the process of authenticating a medicine container 104 prior to administration of a medicine into the body. In at least one embodiment, medicine administration device 102 is preprogrammed with a database 106 of information regarding at least one of a medicine manufacturer, a medicine name, a medicine dosage, a date of medicine manufacture, a medicine lot number, medicine recall information, medicine delivery information, and other manufacturing details. Exemplary medicine containers 104 of the present disclosure can have information printed thereon, etched thereon, contained within a label, contained within a miniature computer chip, etc., whereby said information relates to at least one of the name of the medicine manufacturer, the medicine name, the medicine dosage, the date of medicine manufacture, the medicine lot number, medicine recall information, medicine delivery information, and other manufacturing details. Said information upon or within the medicine container 104 is referred to herein as a cartridge identifier 108. A processor or control unit 110 within medicine administration device 102 is configured to access data within database 106 (or from within database 114, as referred to herein).

In at least one embodiment of the present disclosure, medicine administration device 102 is configured to wirelessly communicate with a network 112, such as via Wi-Fi, Bluetooth, Radio Frequency Identification (RFID), or a optional near-field communication (NFC) mechanism or proprietary protocol. Said network 112 can therefore provide access to data within a database 114 housed within or accessible by an external system 116, such as a static database 114 or a dynamic database 114, whereby said database 114 contains information regarding at least one of a medicine manufacturer, a medicine name, a medicine dosage, a date of medicine manufacture, a medicine lot number, medicine recall information, medicine delivery information, and other manufacturing details. For dynamic database 114 use, such as in the case of the database 114 used by the medicine administration device 102 and/or its code is obsolete, for example, the database 114 used by the medicine administration device 102 and firmware or code, as well as the configuration of the medicine container 104, could be updated over-the-air (OTA) with new firmware, configuration, and data.

In use, for example, a user may position a medicine container 104 within a medicine administration device 102, and if the medicine administration device 102 is configured to communicate with a network 112, the medicine administration device 102 can obtain information regarding the medicine container 104, such as one of the name of the medicine manufacturer, the medicine name, the medicine dosage, the date of medicine manufacture, the medicine lot number, medicine recall information, medicine delivery information, and other manufacturing details, and then compare that information to information from within the database 114 accessed via the network 112 (or database 106 when only database 106 is relied upon by medicine administration device 102). In at least one embodiment, and should the data from the database 114 or database 106 regarding the medicine and the medicine container 104 authenticate the medicine container 104 (by way of comparing the information regarding the medicine container 104 with the information from the database 114 or database 106), then the medicine administration device 102 would be operable to deliver the medicine from the medicine container 104. Should the data from the database 114 or database 106 regarding the medicine and the medicine container 104 fail to authenticate the medicine container 104 (by way of comparing the information regarding the medicine container 104 with the information from the database 114 or database 106), then, in at least some embodiments, the medicine administration device 102 would not be operable to deliver the medicine from the medicine container 104. In other embodiments, the medicine administration device 102 would still be operable to deliver the medicine from the medicine container 104, even though authentication had failed, referred to herein as a “bypass option.” Such a bypass option could, in some embodiments, require a user to request the bypass, whereby the system 100 ultimately approves the bypass, permitting the medicine administration device 102 to deliver the medicine from the medicine container 104. This could occur by way of a communication with database 106 or database 114, whereby said database 106, 114 confirms the bypass request. In other embodiments, a user could still operate medicine administration device 102 to deliver the medicine from the medicine container 104 after learning that authentication had otherwise failed. Such “bypass” options allow, for example, medicine to be delivered from medicine container 104 in instances of emergency or otherwise, with the knowledge that the medicine container 104 could not be authenticated as referenced herein.

Administration devices 102 of the present disclosure therefore comprise a receptacle 200, such as shown in FIG. 2, for receiving at least part of a medicine container 104, and a delivery mechanism 202, such as a needle, configured to deliver medicine from medicine container 104 to a mammalian recipient.

As part of an exemplary authentication process, medicine administration device 102 will consider data from database 106 or database 114 and compare said data to data from medicine container 104. If, for example, medicine administration device 102 determines that the medicine contained within medicine container 104 is not a proper medicine to be injected, then, in at least some embodiments, medicine administration device 102 will not operate to deliver said medicine. If, for example, medicine administration device 102 determines that the medicine contained within medicine container 104 is expired or recalled, then medicine administration device 102 will not operate to deliver said medicine. In embodiments utilizing the “bypass option” as referenced herein, a user could use the medicine administration device 102 to deliver medicine from the medicine container 104 even though authentication had failed.

In at least one embodiment, medicine administration device 102 is operable to deliver a dose of the medicine from a medicine container 104 when at least one type of information is used to authenticate the medicine container 104, such as information selected from the group consisting of at least one of a medicine manufacturer, a medicine name, a medicine dosage, a date of medicine manufacture, a medicine lot number, medicine recall information, medicine delivery information, and other manufacturing information. In other embodiments, medicine administration device 102 is operable to deliver a dose of the medicine from a medicine container 104 when at least two, three, four, or more types of information are collectively used to authenticate the medicine container 104. In various embodiments, medicine administration devices 102 are configured so not to be able to inject a counterfeit medicine from a counterfeit medicine container 104 because said medicine container 104 did not meet the applicable authentication standard(s).

In various embodiments, a feedback loop applies to the authentication process. For example, if a medicine container 104 has a unique identifier, and said cartridge passes authentication, then medicine administration device 102 can deliver medicine from said medicine container 104. That particular medicine container 104 with the unique identifier would then be used to update the data within the applicable database (database 106 or database 114) so that, for example, a counterfeit medicine container 104 repeating said unique identifier would not subsequently pass authentication.

In various embodiments, end-to-end encryption is used in the authentication process. For example, information from a medicine container 104 could be encrypted, and that encrypted information could be compared to data provided within database 106 or database 114, and if it is properly authenticated, then an encrypted confirmation could be provided to the medicine administration device 102 so that said device 102 could be used to deliver medicine from medicine container 104.

In some embodiments, a particular medicine container 104 may contain more than one dose of medicine. For example, if a medicine container 104 contains four (4) doses of medicine, then upon authentication the medicine administration device 102 could deliver a first dose of medicine, and database 106 or database 114 would be updated to identify that the first dose has been delivered and that only three (3) doses remain. Upon administration of the remaining three (3) doses, no further authentication can be made relative to said medicine container 104, and medicine administration device 102 would not operate to deliver any further medicine from medicine container 104.

Further, and in various embodiments, system 100 is configured to only deliver medicine when the time is appropriate. For example, if a particular medicine container 104 contains multiple doses of medicine, and if the medicine can only be administered every eight (8) hours, then medicine administration device 102 could deliver a first dosage of medicine, after authentication, but would then not be operable to deliver a second dosage until eight (8) hours has elapsed from the administration of the first dosage.

In some embodiments, system 100 is configured to only deliver medicine if it has been properly temperature controlled. For example, if a particular medicine container 104 contains a medicine that needs to be maintained at a particular temperature, then part of the authentication process would be to identify a temperature-based piece of information from medicine container 104, such as a color-shifting element that changes color if a particular temperature has been exceeded, for example. If medicine administration device 102 authenticates the medicine name, manufacturer, etc., but if the temperature information identifies that the medicine has been stored in or exposed to an excessive temperature, then, in at least one embodiment, medicine administration device 102 would then not be operable to deliver a dosage of medicine from said medicine container 104.

In various embodiments, medicine administration device 102 is configured to update itself by way of obtaining updated information from database 106 or database 114.

### Example 1

A user places medicine container 104 within medicine administration device 102. Administration device 102 operates to identify information from said medicine container 104. Administration device 102 confirms that the medicine is the correct medicine, that the dose is the correct dose, and that the manufacturer is the correct manufacturer. However, medicine administration device 102 identifies that said medicine container 104 is expired (contains an expired medicine), and as such, medicine administration device 102 is not operable to deliver medicine from said medicine container 104.

The medicine container 104 is then removed and replaced with a replacement medicine container 104. Administration device 102 confirms that the medicine is the correct medicine, that the dose is the correct dose, that the manufacturer is the correct manufacturer, and that the medicine is fresh (not expired). As such, and after said authentication, medicine administration device 102 is then operable to deliver medicine from said medicine container 104.

### Example 2

A user places medicine container 104 within medicine administration device 102. Administration device 102 operates to identify information from said medicine container 104. Administration device 102 confirms that the medicine is the correct medicine, that the dose is the correct dose, but cannot confirm that the manufacturer is the correct manufacturer. As such, medicine administration device 102 is not operable to deliver medicine from said medicine container 104.

### Example 3

Three main companies provide insulin to the U.S. market, namely Eli Lilly & Company, Sanofi, and Novo Nordisk. By way of example, medicine administration device 102 may receive a medicine container 104 containing insulin, but would only operate to administer said insulin if authentication of the manufacturer is made by way of comparing manufacturer data from the medicine container 104 with manufacturer data contained within database 106 or database 114. If one or both of said databases only identify Eli Lilly & Company, Sanofi, and Novo Nordisk as suitable manufacturers, and the medicine container 104 identifies a different manufacturer, then medicine administration device 102 would not be operable to deliver said insulin from medicine container 104.

In addition to counterfeiting detection, the device could verify formulation specificity to ensure the insulin type is appropriate for a single patient's use (e.g., rapid-acting vs. long-acting), or to handle multi-user authentication. For example, an insulin package may be authentic, yet the contents may have been substituted, diluted, or replaced. This also applies to other medicine categories such as GLP-1 receptor agonists used in weight loss, where packaging may be legitimate but the internal container falsified. Therefore, additional layers of validation may be required to confirm that the internal medicine container aligns with external labeling and authentication data.

Bypass options, as referenced herein, can apply to the examples above or other examples.

While various embodiments of devices, systems, and methods for using the same have been described in considerable detail herein, the embodiments are merely offered as non-limiting examples of the disclosure described herein. It will therefore be understood that various changes and modifications may be made, and equivalents may be substituted for elements thereof, without departing from the scope of the present disclosure. The present disclosure is not intended to be exhaustive or limiting with respect to the content thereof.

Further, in describing representative embodiments, the present disclosure may have presented a method and/or a process as a particular sequence of steps. However, to the extent that the method or process does not rely on the particular order of steps set forth therein, the method or process should not be limited to the particular sequence of steps described, as other sequences of steps may be possible. Therefore, the particular order of the steps disclosed herein should not be construed as limitations of the present disclosure. In addition, disclosure directed to a method and/or process should not be limited to the performance of their steps in the order written. Such sequences may be varied and still remain within the scope of the present disclosure.

1. A medicine administration device, comprising:
a receptacle configured to receive a medicine container;
a delivery mechanism configured to deliver a medicine from the medicine container to a mammalian recipient; and
a processor or control unit in communication with a database, the database configured to store information regarding one or more medicines, wherein the processor or control unit is configured to access said stored information;
wherein the device, upon receipt of the medicine container and authentication of the medicine container, is configured to deliver a dose of a medicine from the medicine container via the delivery mechanism.

2. The device of claim 1, wherein said stored information comprises information selected from the group consisting of a medicine manufacturer, a medicine name, a medicine dosage, a date of medicine manufacture, a medicine lot number, and medicine recall information.

3. The device of claim 2, operable to deliver a dose of the medicine from the medicine container when at least one type of information is used to authenticate the medicine container.

4. The device of claim 2, operable to deliver a dose of the medicine from the medicine container when two types of information are used to authenticate the medicine container.

5. The device of claim 2, operable to deliver a dose of the medicine from the medicine container when three or more types of information are used to authenticate the medicine container.

6. The device of claim 1, wherein the database is contained within the device.

7. The device of claim 6, wherein the database can be updated via a network in communication with an external database.

8. The device of claim 1, wherein the database is contained within an external system, the database accessible by the device via a network.

9. The device of claim 8, wherein the medicine administration device is configured to communicate wirelessly with the database contained within the external system.

10. The device of claim 1, wherein if the medicine container is not authenticated, the device would not be operable to deliver the dose of the medicine from the medicine container unless a user of the device bypasses authentication.

11. The device of claim 1, wherein the processor or control unit is configured to authenticate the medicine container.

12. A method of medicine delivery, comprising:
positioning a medicine container within the receptacle of the medicine administration device of claim 1; and
performing authentication of the medicine container.

13. The method of claim 12, further comprising:
administering medicine from the medicine container after the medicine container is authenticated.

14. The method of claim 12, wherein if the medicine container is not authenticated, the device would not be operable to deliver the dose of the medicine from the medicine container unless a user of the device bypasses authentication.

15. The method if claim 12, wherein if the step of performing authentication fails to authenticate the medicine container, the device is not operable to deliver the medicine from the medicine container.

16. The method if claim 12, wherein if the step of performing authentication fails to authenticate the medicine container, the device is operable to deliver the medicine from the medicine container if a bypass step is performed.
